(19) United States
(12) Patent Application Publication
(10) Pub. No.: US 2012/0279499 A1
(43) Pub. Date: Nov. 8, 2012

(54) Negative Pressure Intubation Device

(76) Inventor: Jessica Holland Vickers, Jacksonville, FL (US)

(21) Appl. No.: 13/455,281

(22) Filed: Apr. 25, 2012

Related U.S. Application Data
(60) Provisional application No. 61/482,157, filed on May 3, 2011.

(51) Int. Cl. A61M 16/00

(52) U.S. Cl. 128/200.26

(57) ABSTRACT

This device is designed to be an alternative to the traditional conscious intubation with a cooperative patient. It harnesses the negative pressure created during inhalation and utilizes it to advance a guidewire into the trachea, which then allows for endotracheal intubation. The negative pressure created during a deep inhalation will force air through a hollow delivery tube and carry a Sail-tipped wire through the vocal cords and into the trachea. Once the wire has been deployed, a syringe is attached and air is inflated into the expandible polymer sail. This will expand the sail into a balloon shape, create traction against the walls of the trachea and secure the guide-wire. An endotracheal tube may then be advanced blindly, using the wire as a guide to correct tracheal position and the NPI device removed.

### CROSS REFERENCE TO RELATED APPLICATIONS

This application is a continuation of the provisional patent filed on May 3, 2011. application Ser. No. 61/482,157

No federally funded support or research was involved with the origination of this invention or patent application.

### BACKGROUND OF THE INVENTION

This invention is designed for professionals in the medical field, and is a device specifically for use in securing a patient's airway to allow for ventilatory support. This device provides a novel approach to conscious intubations. Anesthesiologists are often required to perform procedures termed awake, or conscious intubations. This procedure entails multiple steps and allows for patients to continue to ventilate and oxygenate under their own power until an endotracheal tube is in the trachea and the airway is considered secure. This procedure is commonly used for two groups of patients: (1) those who are suspected to be a difficult intubation and unable to be mask-ventilated if rendered unconscious prior to the airway being secured, and (2) patients with suspected neck injuries which renders traditional direct laryngoscopy intubation too dangerous with respect to possible spinal cord damage.

Traditionally, an awake intubation requires many steps to prepare, additional personnel for technical support, and a large and cumbersome fiberoptic scope. Once the patient is prepped and positioned, an experienced anesthesiologist will routinely try to maneuver the fiberoptic scope through the mouth and soft palate, around the epiglottis and through the vocal cords. This is time consuming, often difficult and sometimes even impossible for experienced physicians to successfully accomplish. This approach is wrought with operator error and variable conditions which make this approach inconvenient for the physician and the patient, who is conscious throughout this entire process.

The traditional approach to conscious intubation takes the natural ability of the patient for granted. The average patient can generate considerable force with deep inhalation. With inspiration, the diaphragm moves downward and creates negative pressure in the lungs which drives air through the mouth, vocal cords and trachea. The typical patient can move 400-1000 milliliters of air into their lungs in one deep breath. My invention is novel in that it utilizes the patient's natural inspiration to help secure the airway during a conscious intubation. The patient is awake and able to ventilate until an anchor wire is in place and the airway secured. This device also eliminates the need for a large fiberoptic scope which can be difficult to operate and often takes additional personnel to help set up. In contrast, this NPI device takes little to no advanced skill to use and consists of two small pieces.

### BRIEF SUMMARY OF THE INVENTION

This device is designed for awake intubations and consists 2 parts: part 1, a small-gauge flexible wire with a concave “sail tip” on its distal end (NPI wire). This is delivered into the posterior pharynx by part 2, a small curved Delivery tube, which protects the NPI wire and positions it in the posterior pharynx. Once the tube and wire are positioned before the trachea, the patient is then instructed to seal his or her lips around the tube and take a large breath in. The negative pressure created will send air through the delivery tube and vocal cords, carrying the NPI wire into the trachea. The sail-tip will then inflate to secure the wire's location and a standard endotracheal tube can be passed over the wire to complete the intubation process.

### BRIEF DESCRIPTION OF SEVERAL VIEWS OF THE INVENTION PROVIDED

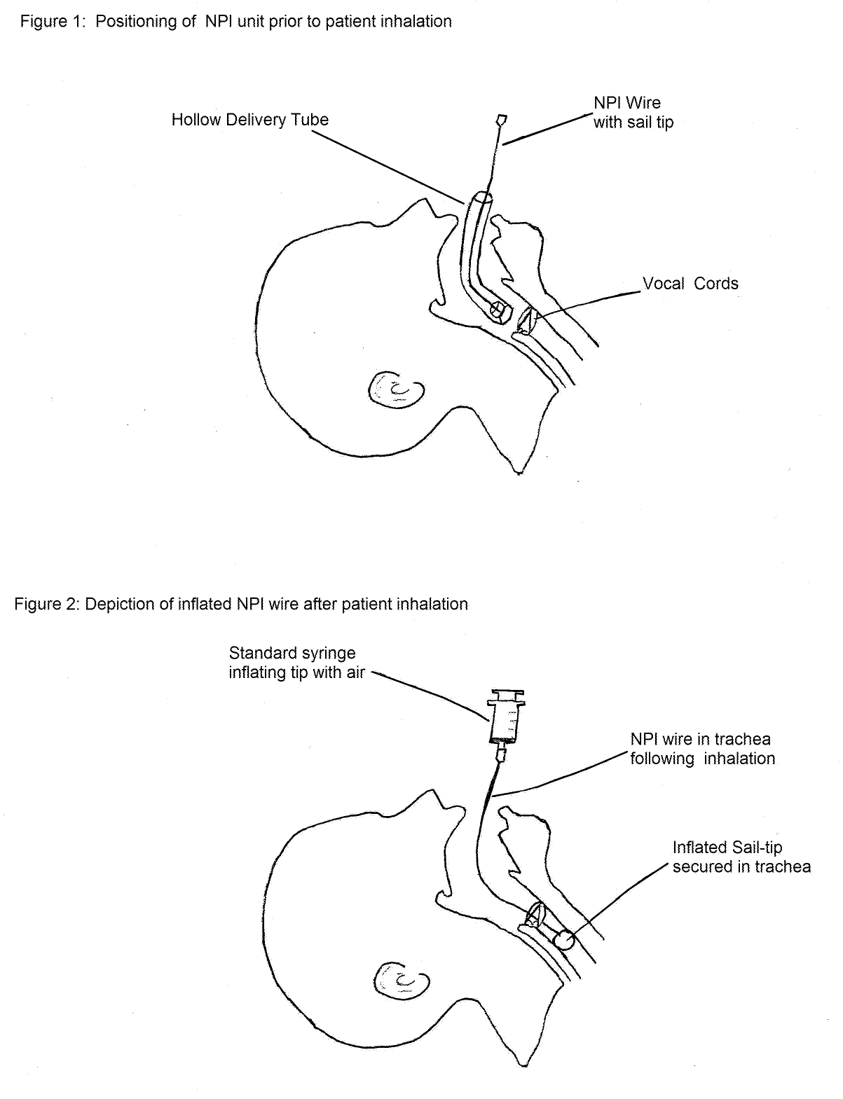

FIG. 1—A sketch of the NPI wire and Delivery tube in correct position just before the patient is asked to inhale.

FIG. 2—The NPI wire has now been carried into the trachea with inhalation. The Delivery Tube has been removed from the mouth and a standard syringe is depicted inflating the “sail-tip” and expanding it into a balloon shape.

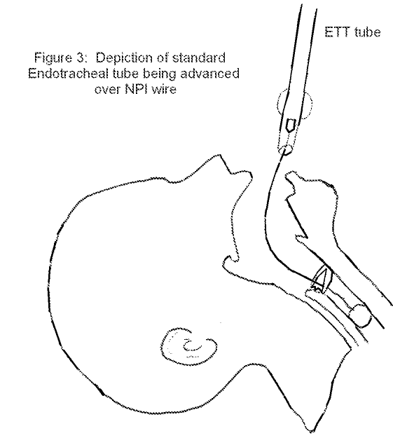

FIG. 3—Represents a depiction of a standard endotracheal tube being advanced over the NPI wire and toward the patient's trachea.

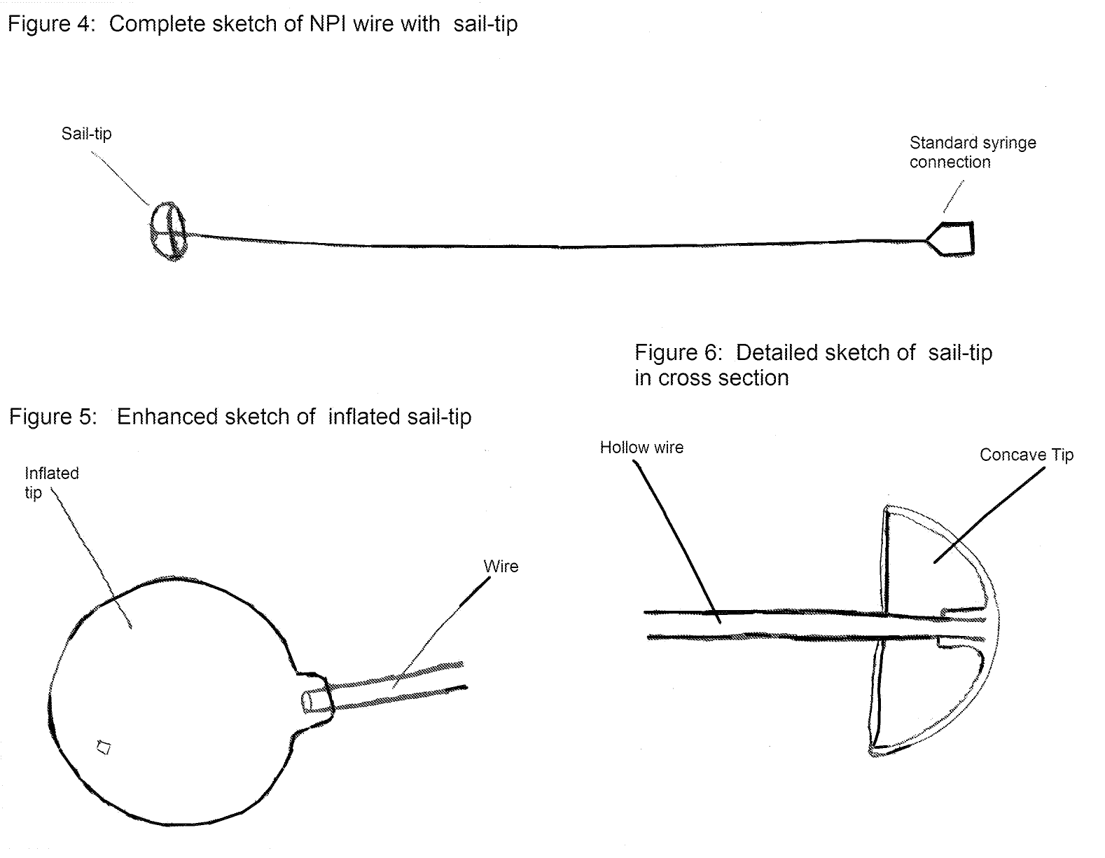

FIG. 4—A more detailed sketch of the NPI wire. Note the concave polymer sail-tip (not inflated) and the standard syringe connection.

FIG. 5—An expanded, detailed image of the inflated sail-tip as it is appears on the NPI wire.

FIG. 6—An expanded, cross-sectional image of the sail-tip prior to inflation. I shows the concave polymer shape in a double layer which will expand into a balloon when inflated with air.

### DETAILED DESCRIPTION OF THE INVENTION

The “sail-tip” will consist of a lightweight polymer, which can inflate against the wall of the trachea and/or bronchus and secure its position. It's design is intended be inflated to triple it's original size. Prior to inflation, the sail-tip is round and convex in shape, much like the shape of a shallow bowl with two layers of polymer with a potential space in-between. When air is injected into the wire, the air fills the potential space in-between the polymer layers and forces expansion into the shape of a balloon. The deflated sail-tip is small enough to pass through abducted vocal cords without impedance (4-5 mm in diameter). This sail-tip is designed to be pulled along through the larynx by air movement during the negative pressure generated by the patient's own diaphragm with inhalation. When the tip is in position, air can be injected through the wire to inflate the sail-tip, causing expansion against the walls of the trachea or bronchus. Once in place, the wire acts as an anchor over which a standard endotracheal tube may be advanced.

The NPI wire is thin, lightweight, and flexible. It is hollow with a luer lock connector on its proximal end that is compatible with a standard syringe. The distal wire has an open distal end that empties into the expandible sail-tip. Once the wire and tip have been inhaled through the vocal cords, air can be injected from a standard syringe through the wire to inflate the sail-tip located in the trachea.

The Delivery tube is a curved small plastic tube. The tube is hollow and round in shape and is manufactured from a stiff polymer which can be manipulated and bent at the distal half. The tube will be approximately 7 mm in diameter to accommodate the NPI wire with and has a curved distal end which can match the curve of the mouth and soft palate. When inserted into the patient's mouth, the tube will extend beyond the tongue and terminate in the posterior pharynx near the vocal cords. The NPI wire will then be placed inside the Delivery tube with the sail-tip being just inside the distal end of the delivery tube. This allows for ideal positioning from which the NPI wire will be pulled through the larynx during active inhalation.

Steps required to use the invention properly.
1. Use local anesthetic in the oropharynx and position the patient as for any other conscious intubation.
2. Place the hollow delivery tube in the patients mouth.
3. Insert the NPI wire into the Delivery tube.
4. Intruct the patient to seal his/her lips around the tube and take a large breath in through the tube.
5. Watch the NPI wire to confirm movement downward during inhalation, then connect the syringe and inflate the NPI wire with air to secure the sail tip against the trachea walls.
6. Remove the Delivery tube out of the mouth.
7. Insert a standard endotracheal tube (ETT) over the wire and advance the ETT along the wire and into the trachea.
8. Deflate the NPI wire and remove it.
9. Confirm proper placement of the ETT.

1. This invention is novel in that it harnesses the patient's own inhalation to assist with endotracheal intubations. No other device on the market uses the negative pressure generated during a inhalation to assist in the placement of a device into the trachea.

2. The scope of this patent is to protect all future devices and products which are designed to utilize this manner of negative pressure to advance devices into the trachea and bronchus; including but not limited to cameras, endotracheal tubes, and other bronchoscopy and biopsy tools.
